(19) United States
(12) Patent Application Publication
(10) Pub. No.: US 2017/0145365 A1
(43) Pub. Date: May 25, 2017

(54) CELL CULTURING DEVICE AND CLOSED-SYSTEM CULTURE VESSEL

(71) Applicant: Hitachi, Ltd., Tokyo (JP)

(72) Inventors: Takayuki NOZAKI, Tokyo (JP); Masaharu KIYAMA, Tokyo (JP); Masakazu SUGAYA, Tokyo (JP); Guangbin ZHOU, Tokyo (JP); Taku NAKAMURA, Tokyo (JP); Daisuke SUZUKI, Tokyo (JP); Shizu TAKEDA, Tokyo (JP); Yumiko IGARASHI, Tokyo (JP); Koichi TERADA, 100-8282 (JP)

(21) Appl. No.: 15/319,905

(22) PCT Filed: Jul. 29, 2014
(86) PCT No.: PCT/JP2014/069955

§ 371 Date: Dec. 19, 2016

(51) Int. Cl. C12M 1/00; C12M 3/00

(52) U.S. Cl. CPC C12M 23/34; C12M 23/48; C12M 29/00

(57) ABSTRACT

A culture which maintains an aseptic state at the time of culturing is achieved, the avoidance of damage to a closed-system culture vessel and of an influence to the cells due to impacts and oscillation is possible during transportation after being manufactured. The closed-system culture vessel 101 has culture vessels 201, 202 for maintaining the cells and/or the culture medium, and a culturing space which is closed by holding members 203, 204 for holding the culture vessels, and the holding member has a semi-open cavity 221 independent of the culturing space. Further, the holding member, by being provided with a lid fixing ring 220 surrounding a connector part 214, is not damaged even when impacts are applied during transportation.

### TECHNICAL FIELD

The present invention relates to a cell incubation technique which cultures cells or tissues with a closed-system culture vessel.

### BACKGROUND ART

The regenerative medical treatment in which the function of organs and the like are recovered using the regenerative tissue manufactured using cells as the raw material is expected as a fundamental medical treatment method for disease for which there have been no medical treatment methods from the past. The object for treatment includes many different types such as the skin, cornea, esophagus, heart, bone, cartilage and the like, and examples of clinical use have also been increasing rapidly. The manufacturing process of the regenerative tissues performs processes such as separating, purifying, amplifying and organizing a biological sample acquired from the patient himself/herself or another person. This process is executed in accordance with Standard Operating Procedure (SOP) satisfying a Good Manufacturing Practice (GMP) that is the standard of manufacturing and quality control of medicinal products and the like in a Cell Processing Center (CPC). Therefore, in order to operate the CPC, a high cost and human resources with specialized culture techniques are required. Additionally, since all manufacturing processes are conducted manually, there is a limit to the production amount of the regenerative tissues. As a result, the low productivity and the high production costs hinder the spread of the regenerative medical treatment, thus, the automation of the culturing operations among the manufacturing processes, specifically, the operations which require labor and cost has been sought. By automating the culturing operations, a reduced workforce and costs are achieved, and mass production becomes possible.

An example of the automated culture apparatus is an apparatus which automatically treats a closed-system culture vessel having a closed space. The closed-system culture vessel is in a state which is constituently connected with flow channel tubes and the like at the time of culturing, and the automated culture apparatus which is in a state which maintains the closed property of the culture space, automatically executes the cell seeding, the culture medium exchange, the microscopic observation and the like. The advantage that the risk of biological contamination is reduced is obtained thereby. After production by the automated culture apparatus, the regenerative tissues are taken from the apparatus in a stored state from the automated culture apparatus to the closed-system culture vessel, and transported to an operating room where the transplantation is performed. At this time, it is necessary to transport the regenerative tissues in a state in which maintains the quality of the regenerative tissues.

As an example of the closed-system culture vessel used in the automated culture apparatus, a closed-system culture vessel and an automated culture apparatus have been disclosed as shown in Patent Reference 1. The closed-system culture vessel has a two layer culture vessel, and flow channels for supplying or discharging the culture medium may be in constant contact therewith. Further, an automated culture apparatus using the closed-system culture vessel in which the culture vessel is one layer has been disclosed in Patent Reference 2.

### CITATION LIST

### Patent Literature

Patent Reference 1: WO 2012/008368

Patent Reference 2: Japanese Unexamined Patent Application Publication No. 2007-312668

### SUMMARY OF INVENTION

### Technical Problem

As shown in Patent References 1 and 2, the configuration of the regenerative tissues which use the closed-system culture vessel within an automated culture apparatus has already been achieved. It is also possible to select the number of layers of the culture vessel in accordance with the cell type, thus, various types of regenerative tissues may be manufactured. These techniques use a closed system culture vessel, and thus, not only is it possible to maintain an aseptic state at the time of culturing, but it is also possible to bring the regenerative tissues to the operating room while maintaining the aseptic state in a stated stored in the closed-system culture vessel even in the transportation of the regenerative tissues after manufacture.

However, during transportation of the closed-system culture vessel after automatic culturing, not only the maintenance of the aseptic state but a countermeasure against impacts and oscillation are necessary. When there are impacts against the closed-system culture vessel during transportation and the culture vessel is damaged, the closed property present during culturing is lost and the aseptic state of the regenerative tissues is lost. In short, the regenerative tissues cannot be used in regenerative medical treatment therapy. Further, by the regenerative tissues receiving oscillations and impacts during transport, there is the danger that an effect will occur in the cells constituting the regenerative tissues. For example, a culture of epithelial cells does not receive oscillations or impacts during culturing, thus, the environment during transportation in which the oscillation and impact occur is different than that of during culturing of the cells. It is preferable that the effect of oscillation and impacts is small.

The closed-system culture vessels disclosed in Patent References 1 and 2 can realize the maintenance of an aseptic state at the time of culturing, but to stably perform the regenerative medical treatment therapy, furthermore, a function for avoiding damage to the closed-system culture vessel and an influence to the cells due to impacts and oscillation during transportation is necessary. By the mounting of such a function, the maintenance of the quality of the regenerative tissues is possible not only during culturing but also after culturing.

The object of the present invention, taking such problems into consideration, is to provide a cell culture apparatus for realizing a culture which maintains an aseptic state, and to make it possible to avoid damage due to impacts and oscillation and the influence to cells during transportation after culturing, and a closed-system culture vessel.

### Solution to Problem

To attain the aforementioned object, the present invention provides a cell culture apparatus of a configuration provided with a culture vessel and a holding member for holding the culture vessel, wherein the culture vessel and the holding member forma culturing space which is closed except for a connector part which performs the feeding of a liquid or gas which are necessary for the culturing, and the holding member uses the closed-system culture vessel having a space independent from the culturing space to perform the cell culturing, and cushions impacts against the closed-system culture vessel by the independent spaces.

Further, to attain the aforementioned object, the present invention is provided with a cell culture apparatus of a configuration provided with the culture vessel in which the cells are held and the holding member for holding the culture vessel, wherein the culture vessel and the holding member form a culturing space which is closed except for a connector part which performs the feeding of a liquid or gas which are necessary for the culturing, and the holding member has a space independent of the culturing space, and cushions the impact due to the independent space.

### Advantageous Effects of Invention

The present invention can realize a culture which maintains an aseptic state at the time of culturing, and the avoidance of damage to the closed-system culture vessel and an influence to the cells due to impacts and oscillation during transportation after manufacture is possible.

### BRIEF DESCRIPTION OF DRAWINGS

FIG. 1 is a diagram for showing a configuration of the automated culture apparatus according to a first embodiment.

FIG. 2A is a cross-sectional view for showing a configuration example of the closed-system culture vessel according to the first embodiment.

FIG. 2B is an assembly view for showing a configuration example of the closed-system culture vessel according to the first embodiment.

FIG. 2C is a top view for showing a configuration example of the closed-system culture vessel according to the first embodiment.

FIG. 2D is a perspective view for showing a configuration example of the closed-system culture vessel according to the first embodiment.

FIG. 3 is a drawing showing an example of the path circuit including the closed-system culture vessel according to the first embodiment.

FIG. 4 is a drawing showing an example of the control mechanism of the cell culture apparatus according to the first embodiment.

FIG. 5 is a drawing showing an example of the flow at the time of operation of the cell culture apparatus according to the first embodiment.

### DESCRIPTION OF EMBODIMENTS

Preferred embodiments of the present invention will be described below in conjunction with the drawings. Note that, the same reference numeral in different drawings indicates the same feature.

### First Embodiment

The first embodiment is an example of a cell culture apparatus of a configuration provided with a culture vessel and a holding member for holding the culture vessel, wherein the culture vessel and the holding member form a culturing space which is closed except for a connector part which performs the feeding of a liquid or gas which are necessary for the culturing, and the holding member uses a closed-system culture vessel having spaces independent from the culturing space to perform the cell culturing, and cushions impacts against the closed-system culture vessel by the independent spaces, and a closed-system culture vessel used by the apparatus.

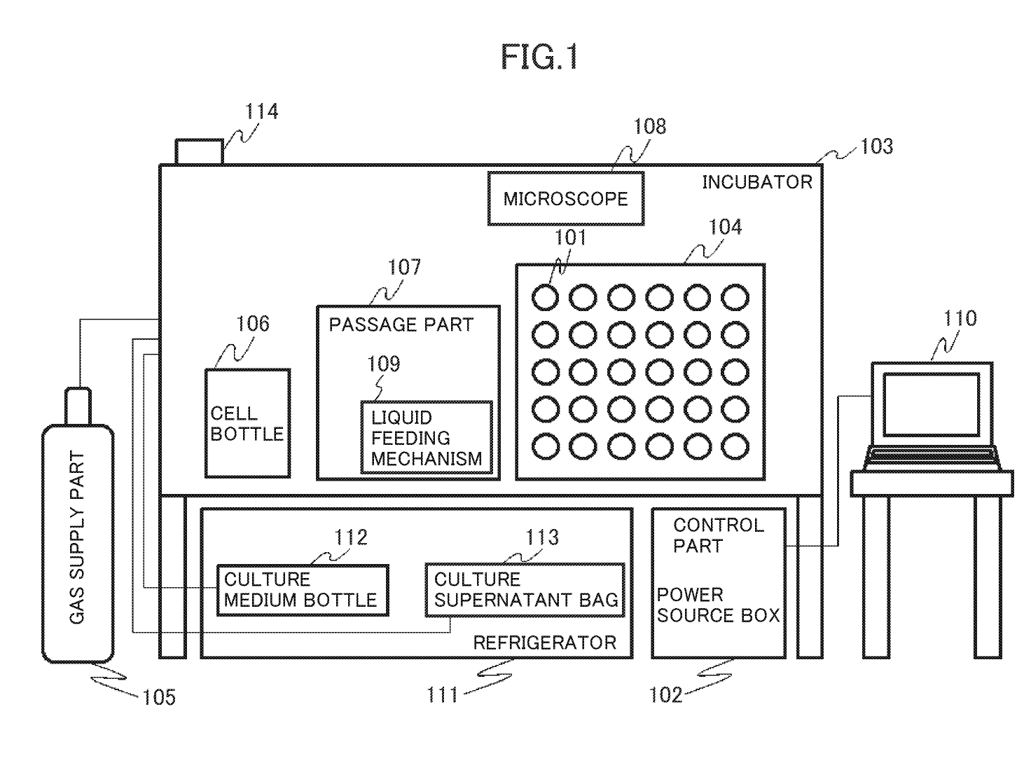

The constitutional elements of the automated culture apparatus for automatically performing culturing using the closed-system culture vessel of the present embodiment will be explained using FIG. 1. The automated culture apparatus includes an incubator 103 which is the space for culturing the cells at 37° C. which is the culturing temperature, a culture medium bottle 106 in which the culture is inserted, and a refrigerator 111 for maintaining the temperature of a culture supernatant bag 113 for collecting a culture supernatant, a gas supply part 105 for supplying air containing 5% CO₂ to the closed-system culture vessel 101, a control part 102 for controlling the automated culture apparatus and the like. The closed-system culture vessel 101 for culturing the cells within a culture vessel part 104 is present in the incubator 103. The number of closed-system culture vessels 101 may be one or a plurality of vessels.

Further, the closed-system culture vessel 101 is constantly connected with the culture medium bottle 112 and the culture supernatant bag 113 via the flow channel tubes and the like which are not shown in the drawings. The cells within the closed-system culture vessel 101 are appropriately observed with a microscope 108. A passage part 107 having a drive system containing an electromagnetic valve for feeding the culture medium, etc., to the closed-system culture vessel 101 and a liquid feeding mechanism 109 such as a tube pump are arranged. Further, the detection for performing concentration control of the gas supplied from the gas supply part 105 is performed by a CO₂.O₂ sensor 114. Note that, a power source box is built within the control part 102, so that it is possible to perform various parameter settings for setting the input and output parts of the control terminal 110 which used a personal computer (PC) and the like as the user interface part.

The automated culture apparatus of the present embodiment performs cell seeding by feeding of the cell suspension to the closed-system culture vessel 101, culturing in which the temperature and the gas environment are maintained, a culture medium exchange for discharging the old culture medium and supplying a new culture medium, the observation of the cell by a microscope 108, and the like. The processes performed by the automated culture apparatus are set as cell seeding, culture medium exchange, culturing, and microscopic observation in the present example, but it is obvious that the invention is suitable even if some of the processes is manually changed.

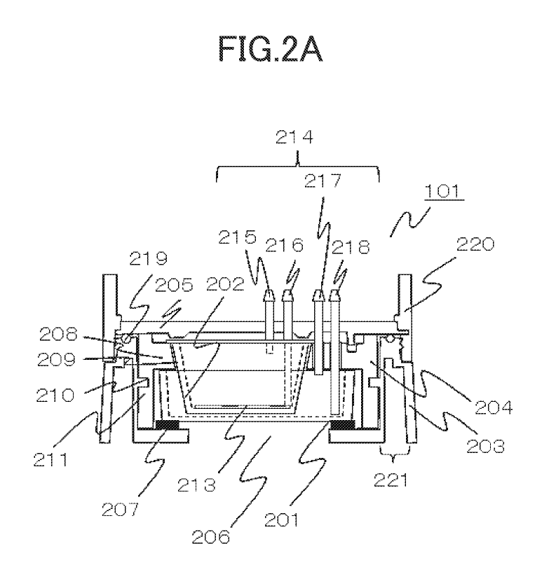

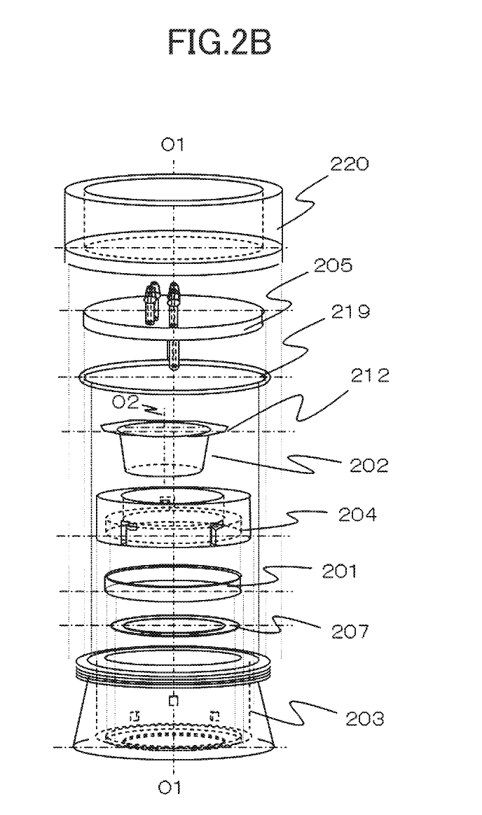

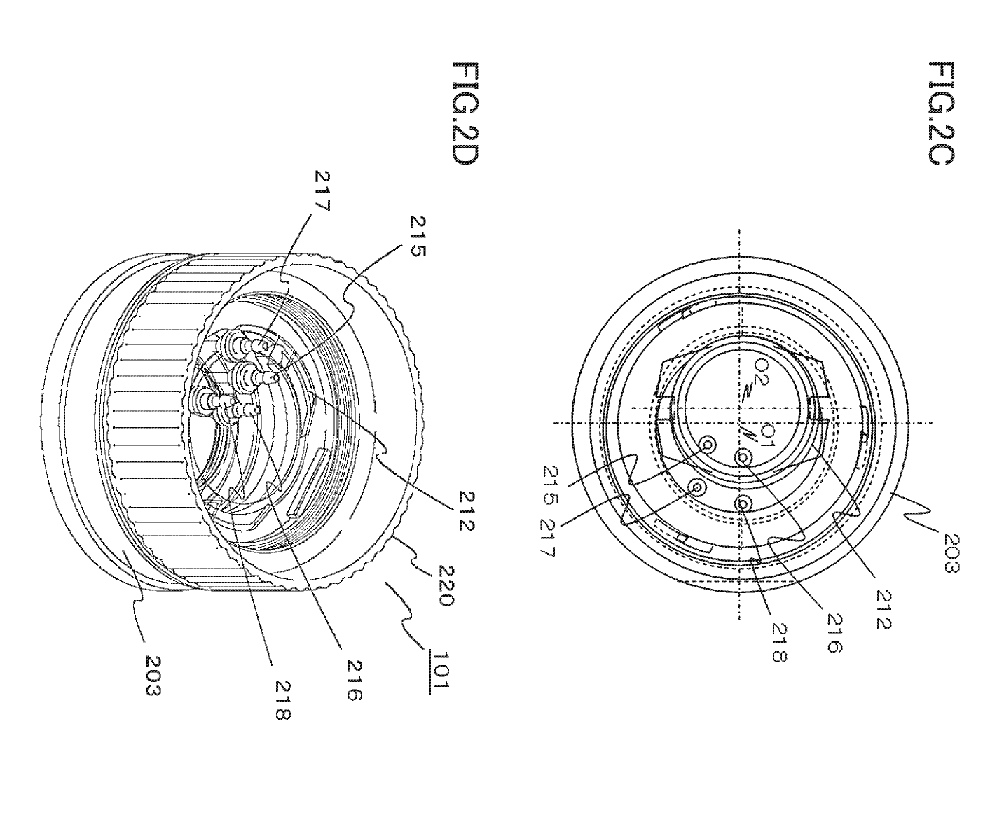

The fundamental constitutional elements of the closed-system culture vessel 101 of the cell culture apparatus of the present embodiment will be explained using FIG. 2A to FIG. 2D. It is necessary that the members of the culture vessel and the holding member for constructing the closed-system culture vessel can be sterilized by a sterilization process. When the material is, for example, polystyrene, the material is subjected to a sterilization operation by an ethylene oxide gas process, a γ-ray radiation process, or the like prior to use, so that sterilization is possible. The aforementioned example used polystyrene, but it is obvious that any compound is suitable as along as the sterilization of the material which is not harmful to the biological sample is possible. Further, it is preferable that no harmful substances are produced and that the substance is of a quality for a medical application.

FIG. 2A shows a cross-sectional view of the closed-system culture vessel of the present embodiment 101, FIG. 2B shows an assembly view thereof, FIG. 2C shows a top view seen from above, and FIG. 2D shows a perspective view from the upper part. The closed-system culture vessel of the present embodiment 101 is comprised of the culture vessel in which the cells are maintained and the holding member for holding the culture vessel, thus, two types of culture vessels 201 and 202 are maintained on the inside of the holding member. The cells and the culture mediums are maintained on the inside of the culture vessels 201 and 202, and it is possible to culture the cells. In other words, the culture vessels and the holding member form the culturing space for culturing the cells.

The culture vessel 201 may be a commercially-available culture dish that is generally used for cell culturing. There are culture dishes manufactured by Becton, Dickinson and Company, Corning Incorporated, and Greiner-bio-one, and the product to be used is not particularly limited. Further, a temperature-responsive culture dish manufactured by CellSeed Inc. can be used. Using a commercially-available culture dish, cellular kinetics such as adhesion, extension, proliferation, and differentiation at the time of cell culture becomes equal. Culture dishes approved as medical equipment for clinical use can be used. Those other than commercially-available culture dishes can be used in accordance with the purpose of the user. The materials are plastic or a glass material, etc., having plasticity and rigidity, such as PC and PS.

Further, the culture vessel 202 is an insertion-type culture vessel, so that the regenerative tissues are present on the inside thereof. The insertion-type culture container may be a commercially-available cell culture insertion container. There are culture containers manufactured by Becton, Dickinson and Company, Corning Incorporated, and Greiner-bio-one, and the product to be used is not particularly limited. Further, the temperature-responsive cell culture insertion container manufactured by CellSeed Inc. can be used. The bottom surface of the cell culture insertion container is of a porous membrane, and has a plurality of holes with a diameter of, for example, about 0.4 μm. Accordingly, the culture medium and liquid factors can be moved between the upper layer and the lower layer. The materials are plastics having plasticity and rigidity, such as PC, PS, and polyethylene terephthalate (hereinafter, abbreviated as PET).

The drawing shows an example in which two types of the culture vessels 201 and 202 are used, and in this case, the culture vessels have two layers. It is possible to use these two types of culture vessels when culturing epithelial cells by the feeder layer method. Further, while not shown in the drawing, it is possible to use only the culture vessel 201, and to make the culture vessel as one layer. In this case, it is possible to use in cultures such as cardiac muscle cells and periodontal ligament cells.

Next, the holding member in the closed-system culture vessel of the present embodiment will be explained in detail. The holding member of the closed-system culture vessel 101 of the configuration shown in FIG. 2A, is mainly comprised of a main container 203, a pressing part 204, and a lid member 205. The shape as a whole, as an example, is a cylindrical shape. The closed-system culture vessel including the culture vessel and the holding member maintains the cells and the culture medium in the culture vessels 201 and 202. The main container 203 has an opening part 206 on the bottom side. The outside of the bottom side of the culture vessel 201 is exposed thereby, and thus, obtains the advantageous point that a clear image can be obtained during cell observation. However, even a configuration which does not have an opening part 206 can be used, and while there is the possibility that a clear image cannot be obtained during cell observation, the advantageous point that the closability further increases can be obtained instead. Further, the main container 203 has a semi-open cavity 221 on the side opposite the side on which the culture vessel 201 is installed. The semi-open cavity 221, in which the culture vessel and the holding member are assembled as the closed-system culture vessel 101, is a space different from the closed culturing space.

In the configuration of the present embodiment, a first elastic body 207 which is a ring-shaped rubber sheet is arranged adjacent to the opening part 206, and the culture vessel 201 is arranged within the main container 203 via the first elastic body 207. The culture vessel 201 and the main container 203 are integrated by the pressing part 204. The pressing part 204 is a cylindrical shape and has a hollow joint portion 208. An inverse truncated conical through-hole 209 that receives the culture vessel 202 which is an insertion-type culture vessel having an inverse truncated conical outer shape is provided in the joint portion 208. The lower surface of the joint portion 208 contacts the entirety of the circumferential upper edge of the culture vessel 201. The pressing part 204 is detachably semi-fixed by fitting along the inner circumference of the main container 203 above the culture vessel 201. Namely, the integration of the pressing part 204 to the main container 203 is completed when a fitting protrusion 210 provided on the inner cylindrical surface of the main container 203 engages with a stepped fitting groove 211 provided on an outer cylindrical surface of the pressing part 204, a rotation angle is applied between the pressing part 204 and the main container 203, and the fitting protrusion 210 engages with the stepped portion of the fitting groove 211.

At this time, the first elastic body 207 located on the bottom surface of the culture vessel 201 is sandwiched between the culture vessel 201 and the pressing part 204 and undergoes elastic deformation by the pressure therebetween, and as a result, the main container 203 and the culture vessel 201 are air tightly sealed. However, when a rotation angle is applied in a direction opposite that of the direction of the integrated pressing part 204, the pressing part 204 is separated from the main container 203. Further, the positional relationship between the main container 203 and the pressing part 204 can be maintained horizontal by three or more sets of the fitting protrusion 210 and the fitting groove 211. Another means such as screwing may be used for fixing the pressing part 204 and the main container 203 horizontally and detachably.

Further, the pressing part 204 can hold the culture vessel 201, and can hold the culture vessel 202 which is an insertion-type culture vessel on the upper flat surface of the joint portion 108. The culture vessel 202 has a flange portion 212 on the bottom surface there of as shown in FIGS. 2B and 2D. A substance permeable membrane 213 is provided on the bottom surface parallel to the flange portion 212. The culture vessel 202 maintains the cells and the culture medium on the substance permeable membrane 213, and culturing is performed.

The lid member 205 of the closed-system culture vessel of the present embodiment 101 has a circular or substantially circular (which will hereinafter be simply called “circular”) planar shape. The lid member 202 has four ports, and these ports collectively connote the following connector part 214. The respective ports are a first port 215 for supplying the culture medium and the like to the culture vessel 202, a second port 216 for discharging the culture medium and the like from the culture vessel 202, a third port 217 for supplying the culture medium and the like to the culture vessel 201, and a fourth port 218 for discharging the culture medium and the like from the culture vessel 201. The connector part 214 is a duct having rigidity and a through-hole passes through the lid member 205 from the upper surface to the lower surface thereof. In other words, the connector part 214 passes through from the outside of the closed-system culture vessel 101 to the inside.

In FIG. 2B, O1 refers to the center of the closed-system culture vessel. This center O1 coincides with the centers of the main container 203, the pressing part 204, the lid member 205, and the culture vessel 201. O2 refers to the center of the culture vessel 202. Namely, the culture vessel 202 of the present embodiment is eccentrically placed in the closed-system culture vessel. This is the result of the optimal placement of the connector part 214 which is explained below. However, the closed-system culture vessel 101 and the culture vessels 201 and 202 are not limited to circular shapes. For example, these vessels may have a shape other than a circle such as a regular hexagon or an ellipse, and may also be placed eccentrically while designating the centers of the shapes thereof as O1 and O2.

In the connector part 214, the first port 215 is placed so that in the culture vessel 202, the opening ends thereof comes slightly below the upper end of the culture vessel 202. The second port 216 is placed so that in the culture vessel 202, it is close to the height of the substance permeable membrane 213 of the culture vessel 202, and, the opening ends thereof are in the vicinity of the outer peripheral position of the substance permeable membrane 213. The third port 217 is placed so that the opening ends thereof are slightly below the upper end of the culture vessel 201. The fourth port 218 is placed so that it in the vicinity of the height of the inside bottom surface of the culture vessel 201, and, the opening ends are placed at a position in the vicinity of the outer edge of the culture vessel 201.

Additionally, the first port 215 and the second port 216 are placed more on one side than the center O2 of the culture vessel 202 (in a region on the right side of O2 in FIG. 2B). Further, the third port 217 and the fourth port 218 are placed more on one side than the center O1 of the culture vessel 201, and, on the same side as the first port 215 and the second port 216 (in a region on the right side of O1 in FIG. 2A). Namely, the connector part 214 is placed on the same side with respect to the center O1 of the closed-system culture vessel, and, in a region on the side opposite to the center O2 of the culture vessel 202. Specifically, the second port 216 and the fourth port 218 are placed on the same side when viewed from the center O1 of the culture vessel 201, or the center O2 of the culture vessel 202 and the same straight line in a radius direction. The lid member 205 is fixed to the main container 203 via a second elastic body 219 which is an O ring. In the present embodiment, the outer circumference of the lid member 205 has a stepped structure having an upper portion and a lower portion having a larger outer diameter than the upper portion in the same manner shown in FIG. 2A.

A groove capable of holding the second elastic body 219 is provided in the upper portion of the main container 203, and the second elastic body 219 is housed in this groove while exposing the contact surface with the lid member 205. A male screw is provided further outside of the groove of the main container 203, and is screwed in a female screw provided in a lid fixing ring 220. The lid fixing ring 220 has an opening part in the center thereof, and has an inner diameter corresponding to the outer diameter of the lower portion of the stepped structure on the outer circumference of the lid member 205. The lid member 205 is fixed to the main container 203 by placing the lid member 205 at the upper surface opening part of the main container 203 of the closed-system culture vessel via the second elastic body 219, and fixing the lid fixing ring 220 to a screw portion on the outer circumference of the main container 203. The lid fixing ring 220 is positioned so that the upper end of the lid fixing ring 220 is higher than the upper end of the connector part 214 at the stage when the lid member 205 and the main container 203 are fixed. Thereby, the connector part 214 is protected by the lid fixing ring 220 from impacts and oscillation. The details are explained in FIG. 2D.

As described above, the culturing space in which the closed-system culture vessel 101 of the present embodiment is formed is air tightly sealed, except for the connector part 214. Further, the closed-system culture vessel 101 is fixed with a screw so that even when the lid 205 is detached from the main container 203 in a horizontal state, little external force is applied to the cells and the culture medium in the main container 203. Therefore, in a culture which used the closed-system culture vessel by the automated culture apparatus, there is a state in which the flow channel tubes and the like are constantly connected to the closed-system culture vessel, thus, the automated culture apparatus is possible in which the cell seeding, the culture medium exchange, the microscopic observation and the like are automatically performed in a state in which the closability of the culturing space is maintained. The advantageous point that the risk of biological contamination is reduced is obtained thereby. Further, after being manufactured by the automated culture apparatus, the regenerative tissues may be taken from the device in a stored state from the automated culture apparatus to the closed-system culture vessel, and may be transported to the operating room where transplantation is to be performed. Therefore, an aseptic state can be maintained by the closability of the culturing space, even from the time of transport to the time the vessel is opened to perform transplantation.

Next, the function by which the closed-system culture vessel avoids the influence of impact and oscillation will be explained using the perspective view of the closed-system culture vessel of the present embodiment 101 shown in FIG. 2D. As stated above, the closed-system culture vessel 101 has a closed culturing space constituted by the culture vessels 201, 202, the main container 203 which is mainly the holding member, the pressing part 204 and the lid member 205. The main container 203 has a semi-open cavity 221 in order to surround this portion. The cavity 221 is in a state in which the lower side of the closed-system culture vessel 101 is open as shown in the cross-sectional view of FIG. 2A. By having a semi-open cavity 221, cushioning is possible when impacts and oscillations are applied to the closed-system culture vessel 101 from the side surface direction. Plastics such as PC, PS and PET and a plastic having rigidity are considered as examples of the material of the holding member, but these have some degree of flexibility.

Furthermore, the portion forming the culturing space is protected in the culture vessels 201, 202 and the holding member by the semi-open cavity 221, and damage in this portion is avoided. Specifically, when impacts are applied in the closed system vessel 101, the impacts are initially applied to the outermost shell of the closed system vessel. Namely, the impacts are initially applied to the portion positioned outside of the semi-open cavity 221 in the main container 203. Therefore, the culturing space positioned on the inside of the semi-open cavity 221 is cushioned. Specifically, it is necessary that the culturing space has closability at the time of transplantation, and that the cleanliness is maintained thereby, but it is possible that the dangers that damage and the like is produced due to impacts and the closability of the culturing space is lost are reduced by the semi-open cavity 221.

Further, as shown in FIG. 2D, the connector part including the four ports 215 to 218 of the closed-system culture vessel surrounds the circumference in the lid fixing ring 220. Specifically, when impacts occurred in the vicinity of the connector part, the impacts are not directly applied to each port 215 to 218 of the connector part, and are initially applied to the lid fixing ring 220. In short, avoiding the influence of impacts in the connector part is possible.

Furthermore, with respect to the impacts and oscillation in the vertical direction, the first elastic body 207 and the second elastic body 219 are arranged within the closed-system culture vessel as shown in FIG. 2A. The first elastic body 207 and the second elastic body 219 are materials such as silicone and rubber. Therefore, when an impact or oscillation is produced in the vertical direction, it is possible to obtain the cushioning effect by these arrangements.

However, the configuration of the present embodiment shown herein can obtain three additional different effects. These will be sequentially described below. Regarding the first effect, the closed-system culture vessel 101 is arranged on the inside of the incubator 103 as shown in FIG. 1, and maintained in the incubator at a temperature which is generally 37° C. Due to the presence of the semi-open cavity 221, it is possible for the air heated to 37° C. by the incubator can enter to the inside of the semi-open cavity 221. Further, the surface area in the closed-system culture vessel 101 becomes wider due to the presence of the semi-open cavity 221 compared to when the semi-open cavity 221 is not present. Therefore, the thermal conductivity increases. Compared to the case when there is a low shape in the vertical direction which does not have the function for protecting the connector part 214 in the lid fixing ring 220, the surface area in the closed-system culture vessel 101 becomes wide in the same manner as the lid fixing ring 220 protecting the connector part 214. Similarly, the thermal conductivity increases due to the lid fixing ring 220.

As stated above, as a result, the uniformity of the temperature distribution in the closed-system culture vessel 101 and the increase of the thermal conductivity are anticipated. There are two advantageous points obtained thereby. The first point is that it is possible for the temperature within the closed-system culture vessel to uniformly and rapidly reach the culturing temperature (37° C.), thus, it is also expected that the activity of the cells becomes uniform. This provides an increase of the manufacturing reproducibility of the regenerative tissues. The second point is that the temperature distribution within the closed-system culture vessel is uniform, thus, the possibility that condensation is produced is reduced. Specifically, when the temperature of the lid member 205 decreases slightly in the circumference, condensation is produced on the lid member 205. Due to the condensation, there is the risk that the cell image acquired by the microscope will be unclear. Specifically, in the automated culture apparatus, the cell image is automatically captured not by hand, but by a device. Additionally, it is necessary that the acquisition of a cell image cell is captured at the point in time of each culture process, thus, even if attempting to retake photographs when nothing but unclear images can be obtained, if the times are different, separate evaluation results will be obtained. The cell image at a given point in time can only be obtained at that point in time. The above advantageous point is a very important point in the automated culture apparatus.

Regarding the second additional advantageous point, the closed-system culture vessel 101 is made light weight by having a semi-open cavity 221. There is an advantageous point in that the handling of the closed-system culture vessel becomes easy at the time of arranging the closed-system culture vessel before the start of automatic culturing, during transport after automatic culturing, and during transplantation for extracting the regenerative tissues. Additionally, an effect for reducing the materials cost is also obtained due to the presence of the semi-open cavity 221, specifically, when manufacturing the closed-system culture vessel by injection molding.

Regarding the third additional advantageous point, when culturing is performed in a state in which a heat storage material or a heater is arranged in the circumference of the closed-system culture vessel 101, and transported in a state in which a heat storage material or a heater is arranged, the lid fixing ring 220 is a shape having a height in the vertical direction, thus, the extraction from inside of the heat storage material covering the circumference becomes easy. The higher the heat storage material or the heater arranged in the circumference of the closed-system culture vessel 101 is in the height direction, the more the heating effect to the closed-system culture vessel improves. However, if it becomes difficult to extract the closed-system culture vessel from the heat storage material or the heater by the vessel becomes too high, the handling of the closed-system culture vessel becomes difficult, for example, during transportation after automatic culturing, and during transplantation for extracting regenerative tissues. By the lid fixing ring 220 being a shape having a height in the vertical direction, even if the heat storage material or the heater increases, for example, a height in the range of the culturing space of the closed-system culture vessel, it becomes easy to extract the closed-system culture vessel from the heat storage material or the heater by holding the lid fixing ring 220 protruding therefrom. Further, as shown in FIG. 2D, the lid fixing ring 220 is easily held by using an irregular shape on the outer surface, is a configuration in which attaching and removing are easy, and thus, the handling is easy due to this point.

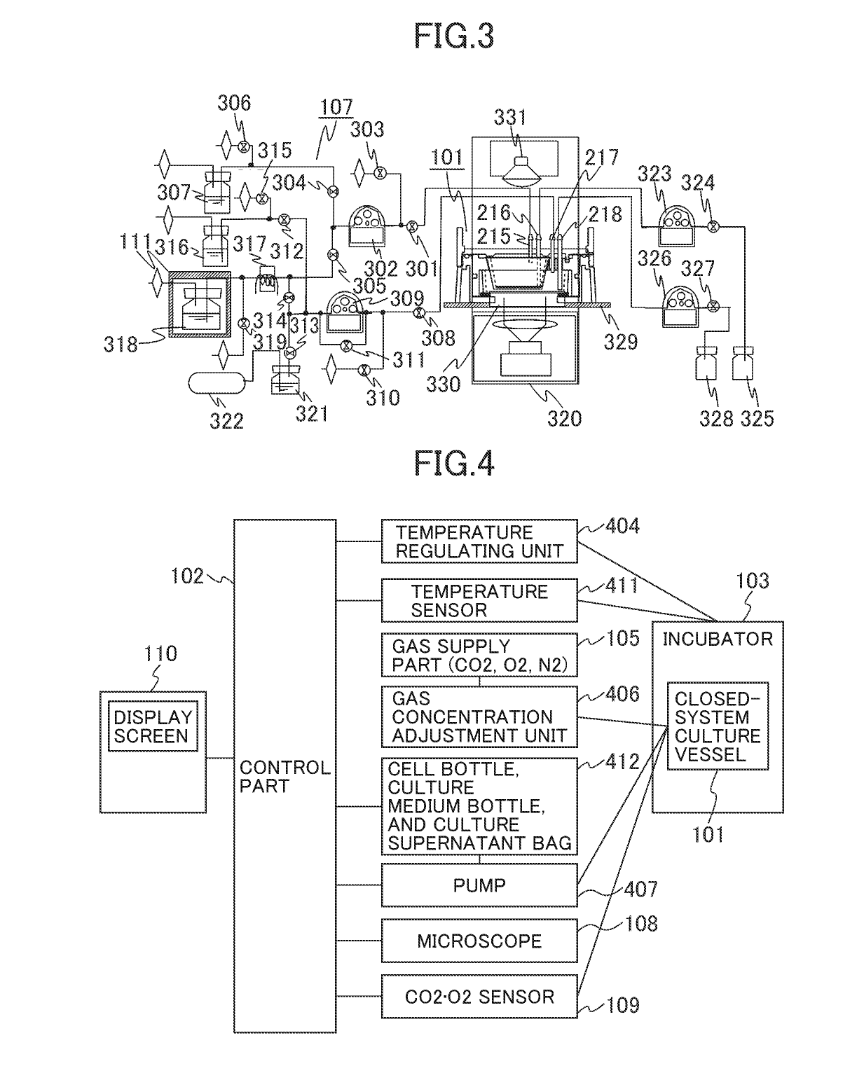

FIG. 3 describes an example of a flow path circuit when the closed-system culture vessel of the present embodiment is used, and the regenerative tissues are manufactured by the automated culture apparatus. The configuration of FIG. 1 is simply shown as the passage part 107 provided with the liquid feeding mechanism 113, but a feeding control part for controlling the feeding relating to the supply or the discharge of the culture medium to the closed-system culture vessel is provided in the flow path circuit of the automated culture apparatus. When supplying gas directly within the closed-system culture vessel, a gas supply control means can also be provided. In the case of a configuration such as when a part of the closed-system culture vessel is made as a gas permeation film, the gas is supplied via the gas permeation film, but in this case, the incubator itself is the gas supply means. An example of the flow path circuit of FIG. 3 is shown in the former configuration.

Further, FIG. 3 shows a microscope observation unit 320 as the observation means used in the automated culture apparatus. Further, in this example, as stated below, two cell bottles 307, 316 are used as the cell bottle 106 of FIG. 1, but this shows an example of the culturing of epithelial cells by the feeder layer method. As stated above, when culturing with a single one-layer culture vessel, only one cell bottle is used. Further, FIG. 3 shows the case when there is one closed-system culture vessel 101, but the automatic culturing of a plurality of closed-system culture vessels simultaneously is possible by disposing the closed-system culture vessels in parallel.

In the flow path circuit example of FIG. 3, a first vessel opening/closing valve 301 of the feeding control part is connected by flow channel tubes to the first port 215 of the closed-system culture vessel 101. The upstream thereof is divided in two, and the first stream is connected to a first pump 302, and the other stream is connected to a first exhaust gas opening/closing valve 303, and furthermore, the upstream thereof is connected to the filter, and the connection port of the other stream of the filter discharges into the atmosphere. The valve mechanism which is used in the first vessel opening/closing valve 301 and the first exhaust gas opening/closing valve 303 is, for example, an electromagnetic valve. The pump which is used in the first pump 302 is, for example, a roller pump. The filter acquires a gas from outside the flow channels to control the atmospheric pressure inside the flow channel, and, a filter of a quality which does not allow passage of particles of a size of, for example, 0.22 μm or more is used.

The upstream of the first pump 302 is divided into two streams, one stream is connected to a first cell opening/closing valve 304, and the other stream is connected to a first culture medium switching valve 305. The upstream of the first cell opening/closing valve 304 is divided into two streams, one stream is connected to a first cell pressure reducing valve 306 and upstream thereof, connected to the filter, and the other stream is connected to a first cell bottle 307. The first cell bottle 307 maintains the cells which are the objects to be cultured in a state suspended in the culture medium. The first cell bottle 307 is provided with an introduction tube and a filter for controlling the atmospheric pressure in the bottle.

To the third port 217 of the closed-system culture vessel, a second vessel opening/closing valve 308 is connected by the flow channel tubes, and the upstream thereof is divided into two streams, one stream is connected in the direction of a second pump 309, and the other stream is connected to a second exhaust gas opening/closing valve 310. Furthermore, the upstream thereof is connected to a filter, and the other connection port of the filter discharges into the atmosphere. The upstream and the downstream of the second pump 309 are each divided into two streams, and the flow channel tubes are connected in parallel so as to by-pass the second pump 309, and a second gas opening/closing valve 311 is connected therebetween. The upstream of the second pump 309 is divided into two streams, one stream is connected to a second cell opening/closing valve 312, and the other stream is divided again into two streams and one is connected to a first gas opening/closing valve 313, and the other one is connected to a second culture medium switching valve 314. The upstream of the second cell opening/closing valve 312 is divided into two streams, one stream is connected to a second cell pressure reducing valve 315, and the other one is connected to a second cell bottle 316. The configuration of the second cell bottle 316 maintains the cells which are the objects to be cultured in a state suspended in the culture medium. The second cell bottle 316 is provided with an introduction tube and a filter for controlling the atmospheric pressure in the bottle.

Both the first culture medium switching valve 305 and the second the culture medium switching valve 314 are, upstream thereof, connected to a preheating mechanism 317, and the upstream thereof is divided into two streams and connected with a culture medium bottle 318 and a culture medium pressure reducing valve 319. The culture medium bottle 318 corresponds to the culture medium bottle 112 of FIG. 1, the culture medium is maintained therein, and the culture medium is refrigerated in a refrigerator 111. At the time of cell culture, after the culture medium is preheated by a preheating mechanism 317, the culture medium is fed to the closed-system culture vessel 101.

The first gas opening/closing valve 313 is, upstream thereof, connected to a humidifying bottle 321, and the humidifying bottle 321 is, for example, upstream thereof, connected to a gas cylinder 322 filled with carbon dioxide gas pressurized at an optimum concentration. In order to prevent the culture medium from undergoing a time-dependent pH change during culturing, for example, by the carbon dioxide gas, it is necessary that the gas in the closed-system culture vessel be changed periodically. Additionally, it is also necessary to prevent the concentration of the culture medium components due to the evaporation of the culture medium. The carbon dioxide gas supplied by the gas cylinder 322 is humidified at an optimal humidity in the humidifying bottle 321, and supplied to the closed-system culture vessel.

The second port 216 of the cell culture vessel is connected to a fourth pump 323 through the flow channel tubes, and downstream thereof, the fourth pump is connected to a fourth vessel opening/closing valve 324, and downstream thereof, the fourth vessel opening/closing valve 324 is connected to an upper layer culture supernatant bag 325. The fourth port 218 is connected to a third pump by the flow channel tubes, and downstream thereof, the third pump is connected to a third vessel opening/closing valve 327, and downstream thereof, the third vessel opening/closing valve 327 is connected to a lower layer culture supernatant bag 328. The upper layer culture supernatant bag 325 and the lower layer culture supernatant bag 328 correspond to the supernatant bag 113 of FIG. 1, the culture supernatant is aseptically collected in the middle of culturing, and the normality of the culture state can be verified by culture medium component analysis. In this case, a separate culture supernatant bottle for aseptically collecting the culture supernatant is arranged in parallel.

A microscope observation unit 320 is arranged below an opening for observation 330 of the stage 329 arranged with the closed-system culture vessel 101. A light irradiation portion 331 which is a part of the microscope observation unit 320 is arranged above the closed-system culture vessel. The microscope observation unit 320 and the light irradiation portion 331 correspond to the microscope 108 of FIG. 1. Further, the stage 329 can adjust the observation location within the closed-system culture vessel by an up/down drive device of the microscope observation unit 320.

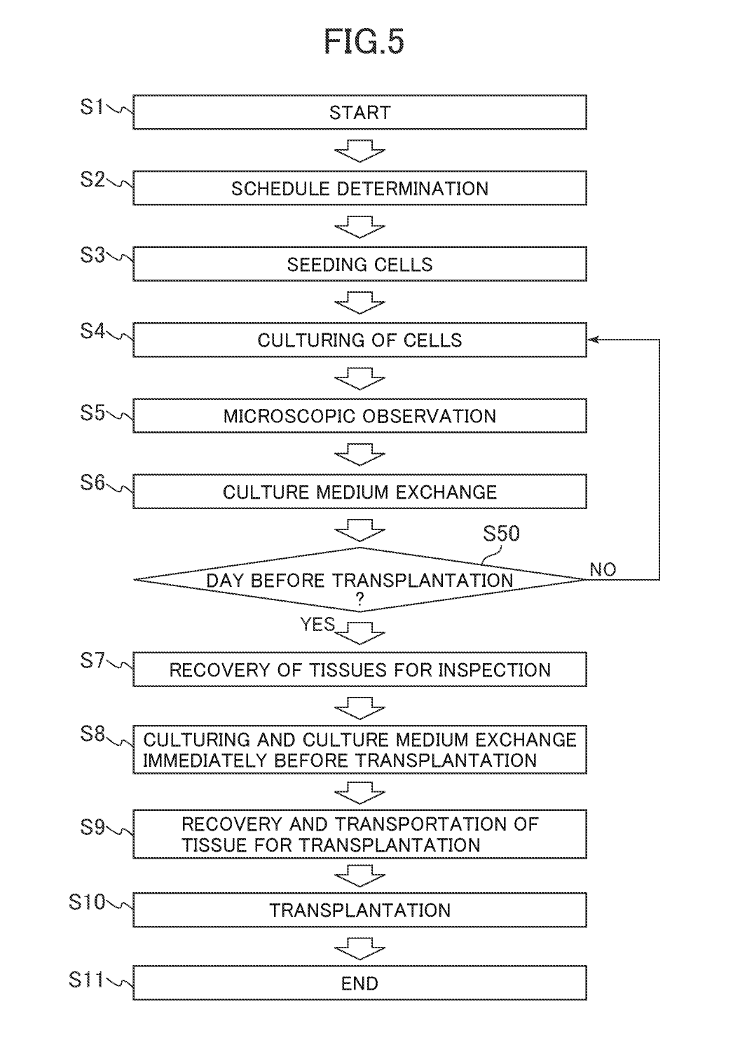

FIG. 4 is a block diagram explaining the functional configuration of the automated culture apparatus containing the closed-system culture vessel of the present embodiment. Each of the constitutional elements controlled by the control part 102 is the entire constitution diagram connected to the closed-system culture vessel 101 arranged inside of the incubator 103. However, it is not necessary to state that the element arranged in the incubator 103 is the above-mentioned closed-system culture vessel 101, or is the culture vessel part 104 arranged in the automated culture apparatus. Note that, the cell bottle, the culture medium bottle and a culture supernatant bag 412 correspond to the cell bottle 106, the culture medium bottle 112 and the culture supernatant bag 113 of FIG. 1, but as shown in FIG. 1, it is desirable to arrange the cell bottle in an incubator 103.

In FIG. 4, a temperature regulating unit 404 for controlling the temperature of the incubator 103, the gas supply part 105 and a gas concentration adjustment unit 406 for controlling the gas concentration in the closed-system culture vessel, a pump 407 arranged in the flow path circuit for automatically switching the culture medium in the closed-system culture vessel, the microscope 108 for cell observation and the CO₂.O₂ sensor 114 which is for the purpose of controlling the operation of the respective constitutional elements are connected to the control part 102. It is not necessary to state that the pump 407 corresponds to the pump groups 302, 309, 323, 326 of FIG. 3.

The control part 102, the control terminal 110 and the display screen thereof correspond to the processor and the display screen of the display of a common computer provided with a processor including a CPU (Central Processing Unit), a memory unit, and an input output part including a display and a keyboard, and the like. The control part 102 operates various programs stored in the memory on the CPU. Therefore, the culturing environment in the incubator 103 can be controlled by the temperature regulating unit 404, the gas supply part 105, the pump 407, the microscope 108, the CO₂.O₂ sensor 114, the gas concentration adjustment unit 406, the cell bottle, the culture medium bottle, and the culture supernatant bag 412, and the execution of predetermined culturing processes in the closed-system culture vessel 101 is possible.

The gas concentration adjustment unit 406 does not need to be directly connected to the closed-system culture vessel 101. The gas concentration adjustment unit 406 may have a configuration that the temperature regulating unit 404, the gas concentration adjustment unit 406 and the CO₂.O₂ sensor 114 are connected to the incubator 103. In this kind of configuration, it is necessary to supply the gas to the closed-system culture vessel 101 from the outside of the vessel, thus, cell culturing becomes possible by depositing a transparent thin membrane having gas permeability such as PC, PS, and polymethylpentene in a part of the lid portion of the closed-system culture vessel 101, and making gas exchange inside of the closed-system culture vessel 101 possible.

The series of procedures when using the automated culture apparatus containing the closed-system culture vessel of the present embodiment having the aforementioned function, manufacturing the regenerative tissues, and transporting the tissues after being manufactured is shown in FIG. 5.

<Step S1: Start>

The closed system flow channels including the closed-system culture vessel are arranged in the automated culture apparatus in advance. The closed system flow channels include the closed-system culture vessel 101, the cell bottle 106 containing the cell suspension, the culture medium bottle 112 containing the culture medium, the culture supernatant bag 113, etc., for collecting the culture supernatant, and the flow channel tubes of the flow path circuit which connect the above. After arranging the closed system flow channels, the normality of the arrangement is verified.

Next, the automated culture apparatus is started. It is started by an operator pressing the start switch on an operation unit in the control part 102, or using an input unit of the control terminal 110. However, the inside of the apparatus is a clean environment due to the execution of disinfection or sterilization in advance. It is verified that the internal environment of the automated culture apparatus is appropriate by the operation screen of the display of the control terminal. For example, it is verified that the temperature of the incubator 103 is 37° C. These numerical values are not restricted and the temperature can be chosen from a range of 0° C. to 45° C.

<Step S2: Schedule Determination>

The automatic culture schedule to be executed by the automated culture apparatus is determined. The conditions such as the date, the frequency, the volume of liquid, and the like for performing operations such as cell seeding, culture medium exchange, culture supernatant recovery, gas exchange, microscopic observation, the recovery of tissues for inspection, and the recovery of tissues for transplantation are inputted from the input portion of the control terminal 110.

<Step S3: Cell Seeding>

After appropriately opening and closing the electromagnetic valve, the pump is operated to suck the cell suspension from the cell bottle 106. The cell suspension is supplied to the closed-system culture vessel 101. After seeding is terminated in all of the closed-system culture vessels, an actuator mounted on a culture vessel base on which the closed-system culture vessel is arranged is operated, an inclination is provided to the culture vessel base and oscillated, and the cell distribution is made uniform.

<Step S4: Culturing of Cells>

Immediately after cell seeding, a gas exchange for supplying a predetermined amount of gas to the inside of each culture vessel is performed. The gas exchange is executed even during the culture period at a frequency of many times per day. As an example, the gas to be supplied uses air including a 5% CO₂ concentration. The flow amount of the gas to each of the closed-system culture vessels from the gas cylinder is controlled by a gas flowmeter, passes through the humidifying bottle, and is supplied in a state saturated with water molecules. The gas which is not needed after being supplied to the closed-system culture vessel 101 is discharged outside the flow channels via the filter. The filter controls the pressure within the flow channels in accordance with need. A filter of a quality which does not allow passage of particles of a size of, for example, 0.22 μm or more is used.

Then, cells are cultured for a predetermined period of time in a state in which the closed-system culture vessel is left standing horizontally. During the culturing, the temperature is maintained at 37° C. by the incubator. The air inside of the apparatus is constantly agitated by a fan so that the temperature distribution becomes uniform. However, a particle count and a viable cell count measurement device can be mounted in the apparatus, and an improvement of the apparatus stability by monitoring the cleanliness is possible.

<Step S5: Microscopic Observation>

A cell image is obtained using a microscope installed in the automated culture apparatus. Alight source installed in the automated culture apparatus emits light appropriately, and the cells are focused upon and the images are captured. The obtained cell images are stored in a database of a memory portion in the control part 102, are made available for viewing on the display of the control terminal 110 of the automated culture apparatus, and the state of the cell can be appropriately verified by the operator. Further, other than at the time of automatic cell photographing, the microscope is operated manually by the operator in accordance with need, and the observation and the photographing of the cell are performed.

<Step S6: Culture Medium Exchange>

The culture medium exchange is performed during the culture period at a frequency of once every several days. The culture medium stored in the culture medium bottle 112 in the refrigerator at 4° C. is supplied to the preheated bottle and preheated. First, the old culture medium is discharged from the closed-system culture vessel 101. At this time, the closed-system culture vessel is inclined by the actuator and the discharging efficiency is improved. After discharge, the new culture medium is supplied promptly into the closed-system culture vessel. The old culture medium is eventually discharged to the culture supernatant bag 113. The culture supernatant in the culture supernatant bag is recovered according to need, and the growth state of the cells is evaluated by culture medium component analysis. However, the culture medium exchange may be performed by a means for extruding the new culture medium in a state in which the old culture medium is taken out.

<Step S7: Recovery of Tissues for Inspection>

When it is verified in S50 to be the day before the day for transplantation, and when a plurality of the closed-system culture vessels are cultured simultaneously, one of the closed-system culture vessels is recovered for inspection. The door of the automated culture apparatus is opened and the flow channel tubes of the closed-system culture vessel 101 for inspection are aseptically removed by a means such as heat welding. The taken out closed-system culture vessels are transported to a safe cabinet or outside the CPC, and an inspection is performed promptly. For example, the number of cells of the biological sample, the survival rate, the expression of specific proteins, and the like are evaluated.

<Step S8: Culturing and Culture Medium Exchange Immediately Before Transplantation>

Culturing by the same operation as in Step S4 is performed. Moreover, immediately before performing Step S9, the culture medium exchange by the same operation as in Step S6 is performed. The microscopic observation by the same operation as in Step S5 is also performed according to need.

<Step S9: Recovery and Transport of Tissue for Transplantation>

When it is determined as a result of the evaluation in Step S7 that the regenerative tissues are suitable for transplantation, the biological samples are recovered and used for regenerative medical treatment. In the same manner as S7, the closed-system culture vessel is aseptically separated from the closed system flow channels and separated from the incubator 103. A carrying procedure to a safe cabinet is performed according to need.

The closed-system culture vessel is stored in a conveyance container for short distances or long distances by a shipping chamber. Influences such as that of the temperature, the pressure, and impacts over the entire distance during transport are avoided by using heat storage materials, an airtight vessel, packaging and the like. A conveyance container is carried outside of the CPC in this state, and is transported by a means such as by motor vehicles, trains, airplanes, or by hand to the operating room in accordance with need.

Before the treatment in an operating room, cell observation is performed by a microscope as an acceptance inspection in accordance with need. When transporting over a short distance, it is assumed that the state immediately before transportation will not change much, and thus, the inspection does not need to be performed depending on the decision of the operator.

<Step S10: Transplantation>

After arriving at the operating room, the regenerative tissues are taken from the closed-system culture vessel. When opening the vessel, there is the possibility that there are organisms such as bacteria and particles adhering to the outside of the closed-system culture, and thus, the closed-system culture vessel is opened aseptically in order to maintain the cleanliness within the closed-system culture vessel.

<Step S11: End>

The closed system flow channels which are used in culturing are removed. Then, sterilization by a sterilization gas or disinfection by ethanol is performed by an appropriate operation on the inside of the apparatus, to attain a clean state. Various software of the automated culture apparatus is terminated, and the operation of the automated culture apparatus is terminated.

According to the preferred embodiment of the automated culture apparatus containing the closed-system culture vessel of the present embodiment constituted as stated above, it is understood that the culture maintains an aseptic state at the time of culturing, and the avoidance of both damage to the closed-system culture vessel and an influence to the cells due to impacts and oscillation during transportation after being manufactured is possible. As a result, the regenerative medical treatment therapy can be stably performed.

As previously explained, the closed-system culture vessel which is used in the culturing in the cell culture apparatus of the present embodiment has a culture vessel for maintaining cells and/or the culture medium, and a culturing space which is closed by a holding member for holding the culture vessel, the holding member has a semi-open cavity independent of the culturing space, and when impacts are applied to the closed-system culture vessel, cushioning of the impact is possible thereby. Further, the portions forming the culturing space in the culture vessel and the holding member are protected by a semi-open cavity, and it is possible to avoid damage in these portions. Furthermore, the closed-system culture vessel by having a configuration which is positioned in an area surrounded by the lid fixing ring of the holding member, a connector part which normally connects a flow channel tubes and the like to the closed-system culture vessel, avoids the influence of impacts and oscillation to the closed-system culture vessel not only during culturing, but also during transport after culturing so that there is no damage even when impacts are applied during transportation of the closed-system culture vessel, and as a result, the maintenance of an aseptic state in the closed-system culture vessel is also possible after culturing.

Note that, the present invention is not limited to the aforementioned examples, and various modification examples can be included. For example, the embodiments have been described in detail to clearly understand the present invention, and are not always limited to one embodiment including all the described configurations.

Some configurations of a certain embodiment can be replaced with configurations of another embodiment, and configurations of another embodiment can be added to configurations of a certain embodiment. With respect to some configurations of each of the embodiments, other configurations can be added, deleted, and replaced.

Furthermore, the configurations, the functions, the control part, and the like described above that achieve some or all of the configurations, the functions, the processing units, and the like with hardware obtained by design or the like of an integrated circuit and can be achieved with software by creating a program that achieves some or all of the configurations, the functions, the processing units, and the like.

### LIST OF REFERENCE SIGNS

101 closed-system culture vessel
102 control part
103 incubator
104 culture vessel part
105 gas supply part
106 cell bottle
107 passage part
108 microscope
109 liquid feeding mechanism
110 control terminal
111 refrigerator
112 culture medium bottle
113 culture supernatant bag
114 CO₂ and O₂ sensor
201, 202 culture vessel
203 the main container
204 pressing part
205 lid member
206 opening part
207 first elastic body
208 joint portion
209 through-hole
210 fitting protrusion
211 fitting groove
212 flange portion
213 substance permeable membrane
214 connector part
215, 216, 217, 218 first, second, third and fourth ports
219 second elastic body
220 lid fixing ring
221 semi-open cavity
301 first vessel opening/closing valve
302, 309, 326, 323 first, second, third and fourth pumps
303, 310 first and second exhaust gas opening/closing valves
304, 312 first cell opening/closing valves
305, 314 first and second the culture medium switching valves
306, 315 first and second cell pressure reducing valves
307, 316 first and second cell bottles
308 second vessel opening/closing valve
311, 313 second and first gas opening/closing valve
317 preheating mechanism
318 culture medium bottle
319 culture medium pressure reducing valve
320 microscope observation unit
321 humidifying bottle
322 gas cylinder
324 fourth vessel opening/closing valve
325 upper layer culture supernatant bag
327 third vessel opening/closing valve
328 lower layer culture supernatant bottle
329 stage
330 opening for observation
331 light irradiation portion
404 temperature regulating unit
406 gas concentration adjustment unit
407 pump
410 display screen
411 temperature sensor
412 cell bottle, culture medium bottle, and culture supernatant bag

1. A cell culture apparatus comprising:
a culture vessel; and
a holding member for holding the culture vessel,
wherein the culture vessel and the holding member forma culturing space which is closed except for a connector part which performs the feeding of a liquid or gas which is necessary for culturing,
the holding member uses a closed-system culture vessel having a space independent of the culturing space to perform cell culturing, and
impacts to the closed-system culture vessel are cushioned by the independent space.

2. The cell culture apparatus according to claim 1,
wherein the independent space is a semi-open cavity, and the gas is free to move to and from the outside of the closed-system culture vessel.

3. The cell culture apparatus according to claim 1,
wherein the independent space makes the temperature distribution within the closed-system culture vessel uniform.

4. The cell culture apparatus according to claim 1,
wherein the independent space reduces the weight of the closed-system culture vessel.

5. The cell culture apparatus according to claim 1 having an elastic material between the culture vessel and the holding member.

6. The cell culture apparatus according to claim 1,
wherein the holding member is provided with a lid member which is penetrated by the connector part.

7. The cell culture apparatus according to claim 1,
wherein the holding member is provided with a ring shape surrounding the connector part.

8. The cell culture apparatus according to claim 7,
wherein the upper end of the ring shape is above the upper end of the connector part.

9. A closed-system culture vessel comprising:
a culture vessel which maintains cells; and
a holding member for holding the culture vessel,
wherein the culture vessel and the holding member forma culturing space which is closed except for a connector part which performs the feeding of a liquid or gas which is necessary for culturing, and
the holding member has a space independent of the culturing space, and cushions the impact due to the independent space.

10. The closed-system culture vessel according to claim 9,
wherein the independent space makes the temperature distribution within the vessel uniform.

11. The closed-system culture vessel according to claim 9,
wherein the independent space reduces the weight of the vessel.

12. The closed-system culture vessel according to claim 9 having the elastic material between the culture vessel and the holding member.

13. The closed-system culture vessel according to claim 9,
wherein the holding member is provided with a lid member which is penetrated by the connector part.

14. The closed-system culture vessel according to claim 13,
wherein the holding member is fixed to the lid member, and is provided with a lid fixing ring surrounding the connector part.

15. The closed-system culture vessel according to claim 14,
wherein the upper end of the lid fixing ring is above the upper end of the connector part.
